(19) United States
(12) Patent Application Publication
(10) Pub. No.: US 2008/0086209 A1
(43) Pub. Date: Apr. 10, 2008

(54) Breast augmentation procedure

(76) Inventor: Melvyn Bircoll, Los Angeles, CA (US)

Correspondence Address: Mark E. Ogram;Ste 203, 7454 E. Broadway, Tucson, AZ, 85710, US

(21) Appl. No.: 11/998,720

(22) Filed: Dec. 1, 2007

Related U.S. Application Data
(63) Continuation of application No. 10/941,175, filed on Sep. 14, 2004.
Application No. 10/941,175 is a continuation of application No. 10/294,238, filed on Nov. 13, 2002, now abandoned.
Application No. 10/294,238 is a continuation of application No. 09/683,442, filed on Dec. 28, 2001, now abandoned.

(51) Int. Cl. A61F 2/12

(52) U.S. Cl. 623/8

(57) ABSTRACT

A medical procedure for the placement and inflation of an implant such as a breast implant. Once an implant site has been identified, a cavity is created and a sheath is inserted thereinto. The sheath contains a pre-sterilized and pre-packaged inflatable implant. The implant is property oriented and then the sheath is withdrawn from site while maintaining the implant within the surgical site. The implant is then inflated via a fill tube and a self-sealing valve. Finally, the fill tube is removed.

This is a continuation of U.S. patent application Ser. No. 10/941,175, filed on Sep. 14, 2004, and entitled “Implant/Insertion Sheath Complex: Inflatable Breast Augmentation for Insertion Through a Small Distal Incision”, which was a a continuation of U.S. patent application Ser. No. 10/294,238, filed on Nov. 13, 2002, and entitled “Implant/Insertion Sheath Complex: Inflatable Breast Augmentation for Insertion Through a Small Distal Incision”, which was a continuation of U.S. patent application Ser. No. 09/683,442, filed on Dec. 28, 2001, and entitled “Implant/Insertion Sheath Complex: Inflatable Breast Augmentation Prosthesis for Insertion through a Small Distal Incision”.

### BACKGROUND OF INVENTION

Breast augmentation is a surgical procedure that has been performed successfully for many years. Traditionally, the procedure was performed with silicone gel prostheses. Recently, however, many doctors have abandoned the silicone gel prosthesis due to the medical-legal implications associated with it. Instead, these doctors use saline-filled inflatable prostheses in breast augmentation procedures.

In the standard breast augmentation procedure, and regardless what kind of implant is actually used, an incision is made on or under the breast. This incision provides access to a retro-mammary space. In the retro-mammary space, the surgeon dissects a “pocket” and then places the implant inside that pocket.

Prior to placing a saline-filled inflatable implant in the retro-mammary pocket he or she has created, the surgeon must remove the implant from its sterile container, fill the implant and test for the “integrity” of the implant (i.e., test to ascertain that there are no holes or other deformities in the implant) by squeezing it in its inflated state. Once the integrity of the implant has been confirmed, the surgeon empties the implant of all fluid and air, rolls the implant into a tightly-rolled “cigar-like” structure, and then forces the rolled implant, frequently with the aid of an instrument, into the pocket in the retro-mammary space. Next, the surgeon fills the implant with the desired quantity of saline through a filling tube with a self-sealing valve. Finally, the surgeon closes the incision and applies a dressing to the incision.

Rolling the implant into a “cigar-like” shape narrow enough to be introduced into the retro-mammary space through a small distal incision is time-consuming and often difficult. It is also the principal cause of damage to the implant, which can result in post-operative deflation of the implant, and the necessity of a second, corrective operation. Another major cause of damage to the implant is the surgeon's forcing the rolled implant through the incision and the subcutaneous tunnel leading to the retro-mammary space. Although devices in the form of plungers, tubes and insertion sheaths have been developed to ease introduction of the rolled implant into the pocket in the retro-mammary space, none of these devices has successfully minimized the surgeon's handling of the implant.

The invention implant/insertion sheath complex is a pre-packaged inflatable breast implant that has been tested, rolled, and encompassed in its own insertion sheath, complete with handle, by the manufacturer before being sterilized and shipped to the surgeon. The invention implant minimizes the surgeon's handling of the implant—the principal cause of damage to implants generally—as a factor in possible damage to any given implant.

### DETAILS OF THE INVENTION

The invention implant/insertion sheath complex is a pre-packaged inflatable breast implant that has been tested, rolled, and encompassed in its own insertion sheath, complete with handle, by the manufacturer before being sterilized and shipped to the surgeon. The implant is prepared (though not necessarily in this order), and has the attributes described, as follows: After manufacture of the individual implant itself, the manufacturer tests the implant for defects and “integrity” using state-of-the-art tooling. The manufacturer then applies vacuum suction or other state-of-the-art tooling to the implant to thoroughly remove all air and fluid from the implant. The manufacturer, again using state-of-the-art tooling, then rolls the implant into a tightly-rolled (e.g., 10 mm. or less in diameter) “cigar-like” structure. The implant has a state-of-the-art self-sealing valve to which a filling tube is attached. The filling tube is long enough to extend from the implant itself (as it will be positioned in the retro-mammary space) to and out of the distal incision (e.g., the umbilicus).

The compressed, rolled implant and attached filling tube are both encased in an “insertion sheath.” The portion of the insertion sheath encompassing the rolled implant (the “implant compartment”) is actually comprised a number of “walls” (e.g., three) which are held in place by “pins” or other mechanisms. The implant compartment is so designed to permit “release” of the insertion sheath upon correct placement of the implant in the retro-mammary space. The portion of the insertion sheath encompassing the filling tube (the “tubular compartment”) is a standard tubular structure, the proximal (i.e., near the incision) end of which (e.g., the “handle”) is marked with indices to show how the implant is to be properly oriented in the retro-mammary space. At the junction of the implant compartment and the tubular compartment is a disc with the approximate diameter of the tube. The disc supports the implant (i.e., acts as a “floor” for the implant) and is itself held in place by a long rod, extending from the disc to and beyond the proximal end of the insertion sheath.

The entire implant/insertion sheath complex is introduced through a distal incision (e.g., the umbilicus) through a subcutaneous tunnel into the retro-mammary space. The leading edge of the implant compartment portion of the insertion sheath is closed and blunt to prevent tissue from entering the insertion sheath during insertion of the implant/insertion sheath complex. This “blocking” function may be accomplished in one of two ways. First, the distal portion of the “walls” of the implant compartment can curve and “meet.” In the alternative, a soft flap attached at one end to one or more of the “walls” of the implant compartment can cover the distal opening (i.e., leading edge) of the insertion sheath. This soft flap attached at one end to one of the “walls” of the implant compartment can cover the distal opening (i.e., leading edge) of the insertion sheath to prevent tissue from entering the insertion sheath upon introduction of the insertion sheath through the incision, into the subcutaneous tunnel and the retro-mammary space. In another embodiment of the invention the covering flap for the leading portion of the insertion sheath can be firm and convex.

Once the implant has been properly oriented in the retro-mammary space, the surgeon releases the implant compartment by removing the pins or other mechanisms holding it in place. This can be done by pulling on a wire or other mechanism that extends down the tubular compartment to the “handle” for easy access by the surgeon. The “walls” of the implant compartment then open on hinges or other similar state-of-the-art mechanisms like the petals of a flower. After the implant compartment is released, the surgeon begins to remove the tubular compartment of the insertion sheath while applying mild pressure to the rod holding the supporting disc (i.e., the “floor” of the implant compartment) and implant in place. This pressure assures that the implant stays in the proper position while the insertion sheath is being removed. After the implant compartment has been completely withdrawn from the retro-mammary space, the tubular compartment and rod (i.e., the entire insertion sheath) is completely removed, leaving only the implant in the retro-mammary space, and the attached filling tube protruding from the proximal incision.

The surgeon fills the implant through the filling tube and self-sealing valve with the desired volume of saline and then removes the filling tube after the inflation.

Use of the disclosed invention implant/insertion sheath complex simplifies the breast augmentation procedure and shortens the time in which the surgeon can perform the breast augmentation procedure.

### DRAWINGS IN BRIEF

FIG. 1 shows an overall labeled diagram of the invention.

FIGS. 2 and 2A show the disk floor and the support rod of the disk floor and the clamp mechanism that holds the support rod in place until it is used.

FIGS. 3, 3A, 3B and 3C show the hinge mechanisms at the junction of the “implant compartment” and the flap covering of the open leading portion of the insertion sheath.

FIGS. 4, 4A, 4B and 4C show the mechanisms for the release of the walls of the “implant compartment”.

### DRAWINGS IN DETAIL

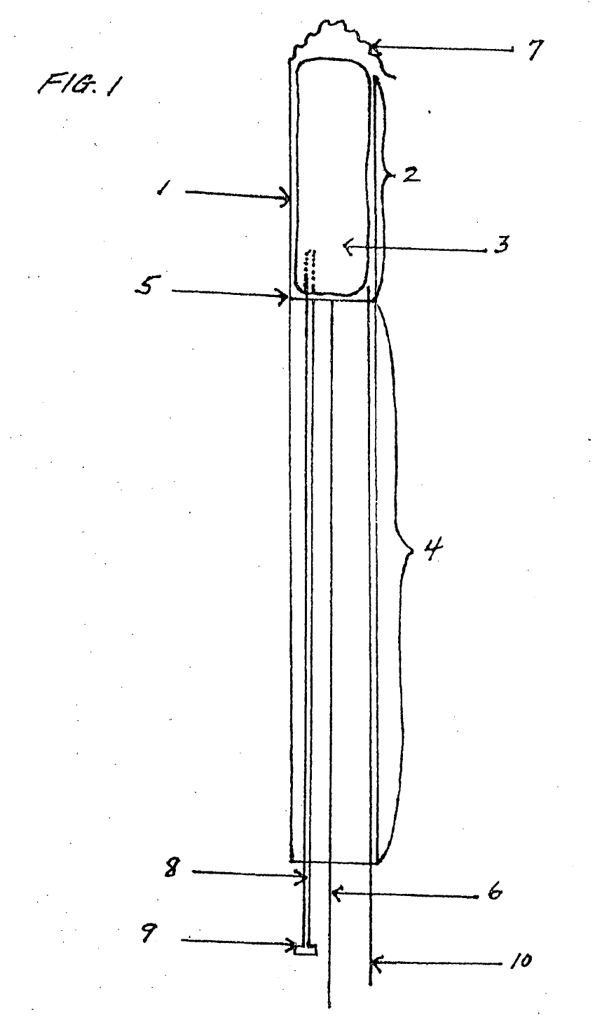

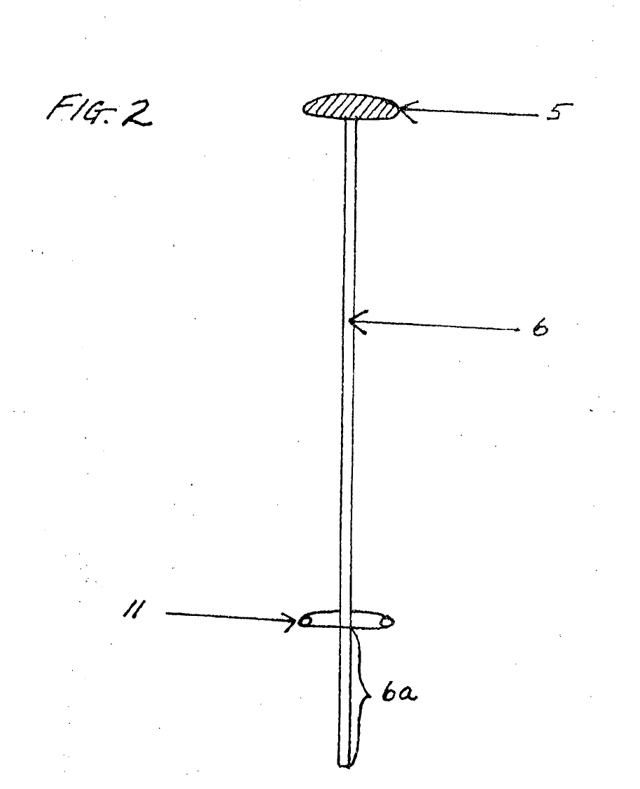

FIG. 1 illustrates an overall labeled embodiment of the invention, Implant/Insertion Sheath Complex: Inflatable Breast Augmentation Prosthesis For Insertion Through A Small Distal Incision.

The “implant compartment” 2 is the distal portion of the Insertion Sheath Complex. The implant compartment 2 may vary in diameter or length depending on the size or nature of the implant it houses. In this embodiment of the invention the implant 3 is a pre-packaged inflatable breast implant that has been tested, rolled, and encompassed in its own insertion sheath 1. The implant compartment 2 is contiguous with the “tubular handle” 4 which may also vary in diameter and length depending on a particular use. The diameter of the tubular handle 4 is always the same as that of the implant compartment 2.

The implant 3 itself is supported by a “disk floor” 5 which has as its' diameter the inside diameter of the implant compartment 2 and the tubular handle 4. Extending downward from the disk floor 5 center is a “support rod” 6 used to the keep the disk floor 5 and implant 3 in position as the sheath is removed. The diameter of the support rod 6 may be small but is must be strong enough and stiff enough to fulfill its' function of support. The support rod 6 must extend beyond the lower end of the tubular handle 4 for a distance slightly greater than the length of the implant compartment 2.

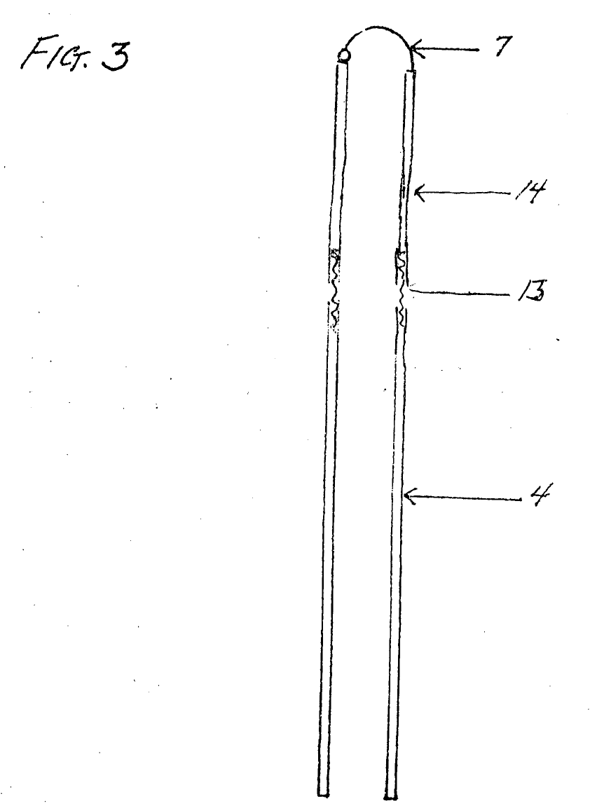

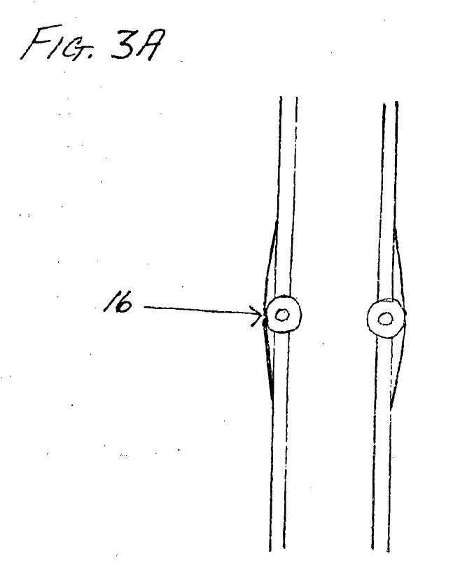

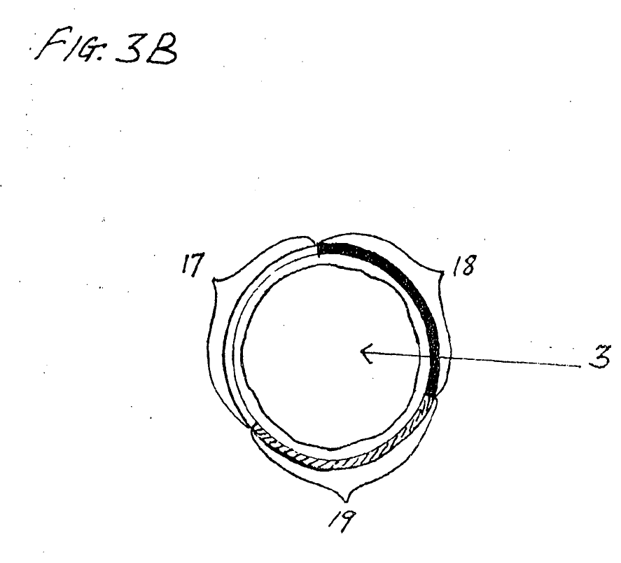

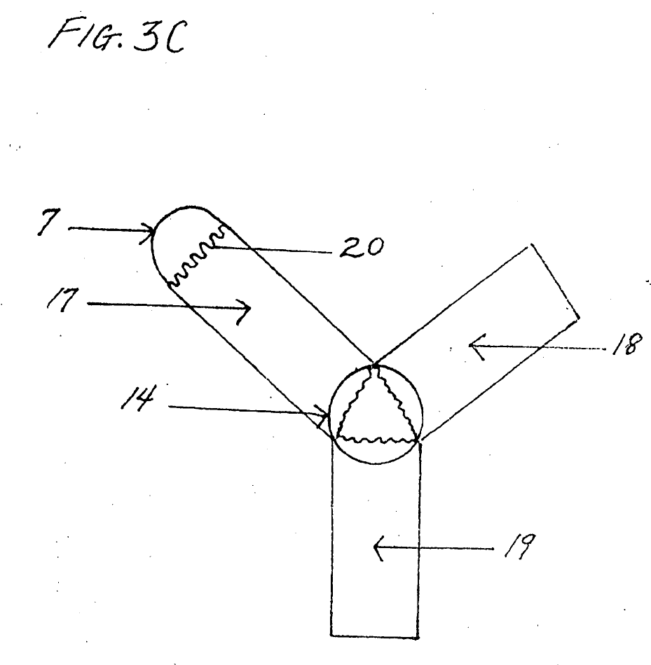

Covering the leading open portion of the invention device is a “covering flap” 7 which prevents any tissue from entering the implant compartment 2 upon insertion of the invention. This covering flap 7 may be a flexible semi-rigid materiel. It may be flat across the opening or dome shaped. It is attached to only one leaf 17 18 19 (FIGS. 3, 3B and 3C) of the wall 14 (FIG. 3) of the implant compartment 2 such that it will not interfere with the opening of the implant compartment 2.

A standard “fill tube” 8 extends from the implant 3 for a sufficient length to come out of the lower opening of the tubular handle 4 and ends in a “filling tube connector” 9 which may be a luer-lock or other type of state of the art connector. The filling tube 8 is attached to the implant 3 at a self-sealing valve. It is through this filling tube that suitable FDA approved liquid may be inserted to fill the implant 3. After insertion of the fluid, the fill tube 8 is removed and the self-sealing valve insures the integrity of the implant 3 at the point of attachment of the fill tube 8.

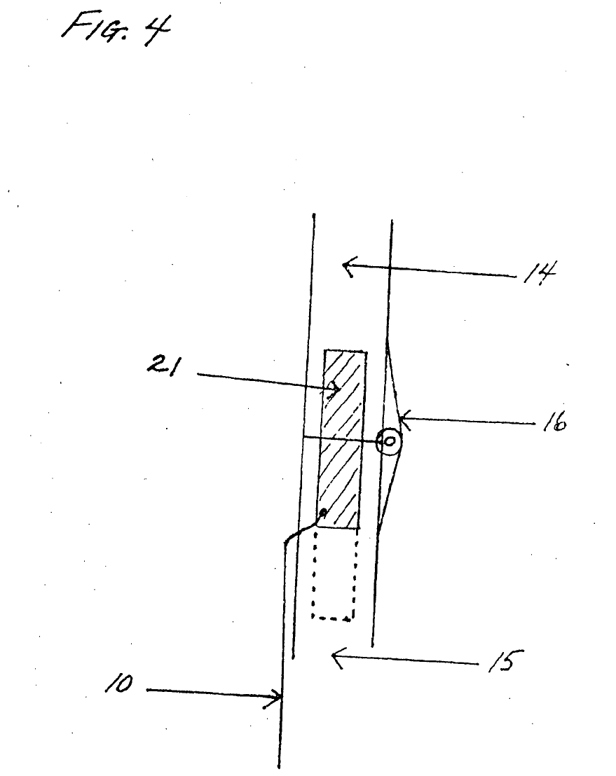

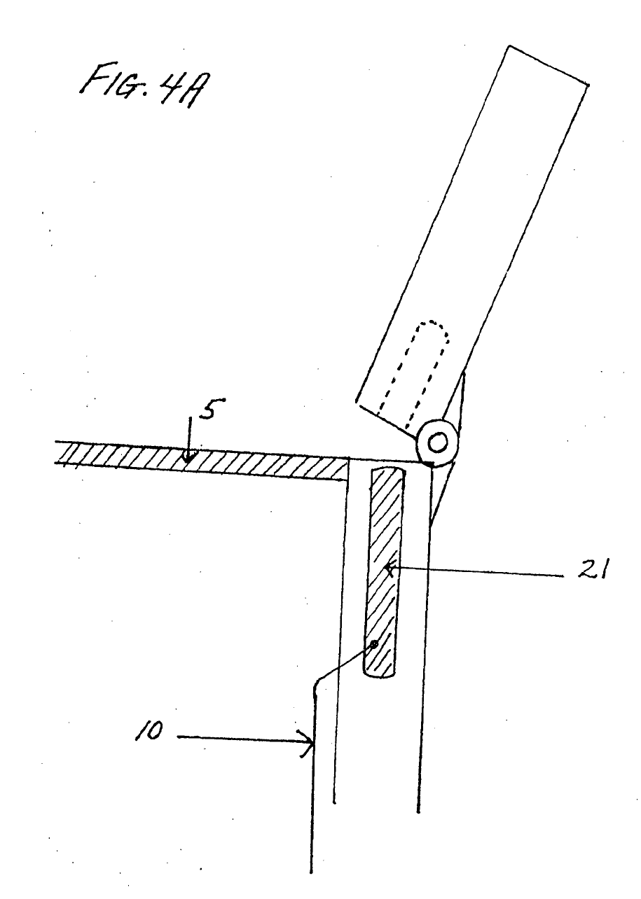

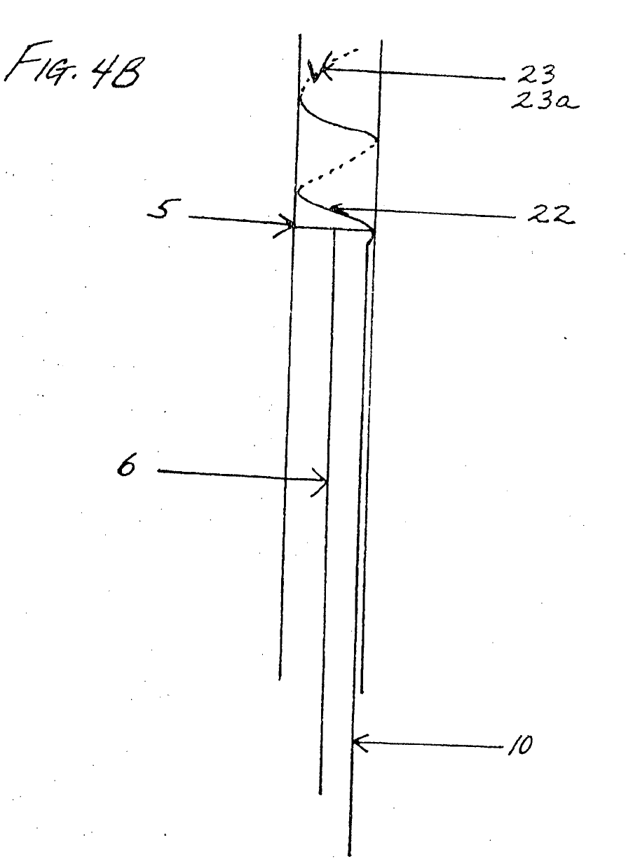

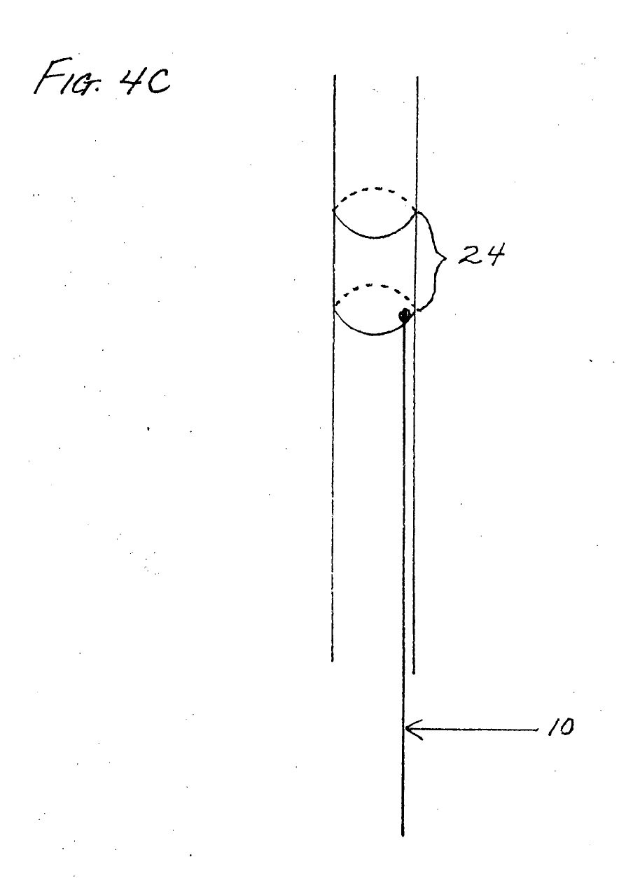

A “pull wire” 10 runs in a groove or in a small tube on the inside of the tubular handle 4. It is attached to release mechanisms (FIGS. 4, 4A, 4B and 4C) at the junction of the implant compartment 2 and the tubular handle 4. The pull wire 10, when activated removes the stabilizing mechanisms and allows for opening of the implant compartment 2.

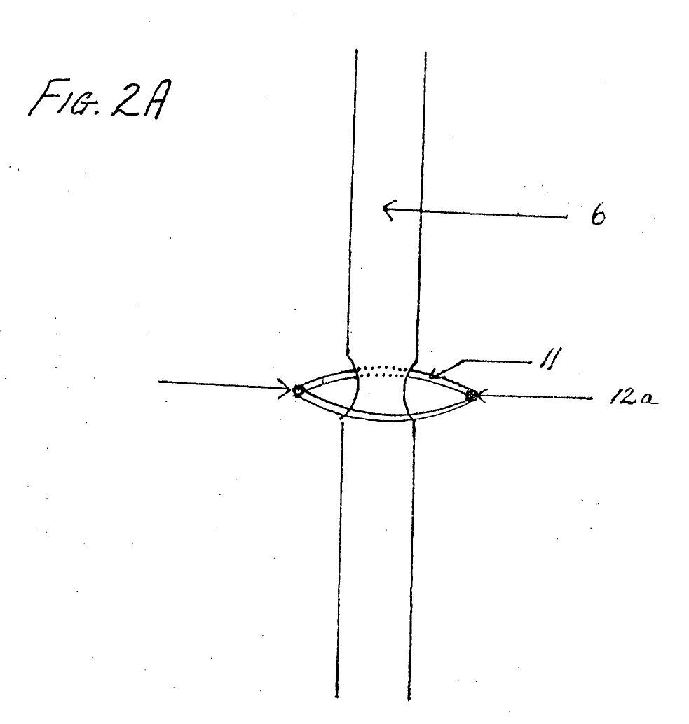

FIG. 2 illustrates the support rod 6, the disk floor 5 of the implant compartment 2 and a stabilizing clamp 11 which clamps around the support rod 6. The stabilizing clamp 11 holds the support rod 6 and thus disk floor 5 in place until such time as it is used in the procedure. The stabilizing clamp is released and the support rod 6 is gently advanced to keep the implant 3 in position as the insertion sheath 1 is removed. The support rod 6 may be of a small diameter but it must have sufficient strength and rigidity to keep the disk floor and thus the implant 3 in proper position. The distal end of the support rod 6A extends beyond the lower end of the tubular handle 4 for a distance slightly greater than the length of the implant compartment 2. This is to enable to support rod 6 to function and hold the implant 3 itself in position as the insertion sheath 1 is withdrawn.

FIG. 2A is an enlargement of the lower portion of the support rod 6. It illustrates simple spring loaded/tension loaded “clamp” 11 which is fasted to the inner surface of the tubular handle 4 by “attachment hinges” 12A 12B. The spring loaded/tension loaded clamp 11 holds the support rod 6 in place when closed and allows movement of the support rod 6 when open. To perform its' function properly, the spring loaded/tension loaded clamp 11 fits around a groove in the support rod 6.

FIG. 3 illustrates the a hinge mechanism at the junction of the implant compartment 2 and the tubular handle 4 and the cover flap 7 for the open leading portion of the insertion sheath 1. This embodiment of the invention demonstrates a “flexible materiel hinge” 13 imbedded in the wall 14 of the implant compartment 2 and the wall 15 of the tubular handle 4 at the junction of these two components.

The cover flap 7 for the open leading portion of the insertion sheath 1 is attached to the distal edge of one component or “leaf” 17 18 19 (FIG. 3B) of the implant compartment 2. In this embodiment of the invention, a soft hinge mechanism is used for the attachment of the cover flap 7 but other flexible fastening methods may be used. In this embodiment of the invention, the cover flap 7 for the open leading portion of the insertion sheath 1 is demonstrated to be a semi-rigid, convex cap. In another embodiment of the invention the cover flap 7 is flat across the open leading portion of the insertion sheath 1. The cover flap 7 may vary greatly in rigidity.

FIG. 3A illustrates an embodiment of the invention with an “external attached hinge” 16 imbedded in the wall 14 of the implant compartment 2 and the wall 15 of the tubular handle 4 at the junction of these two components.

FIG. 3B is a cross section of the invention through the mid-point of the implant compartment 2. It illustrates three (3) leafs 17 18 19 which fit snugly together forming the complete circumference of the implant compartment 2. In order to open properly, there must be a minimum of three (3) leafs 17 18 19 as components of the wall 14 of the implant compartment 2. Other embodiments of the invention may have more than (3) leafs 17 18 19 as components of the wall 14 of the implant compartment 2. The implant 3 is illustrated tightly held in the implant compartment 2.

FIG. 3C is a cross section of the invention at the junction of the implant compartment 2 and the tubular handle 4 with each leaf 17 18 19 open to ninety degrees. In use, each leaf 17 18 19 would open just sufficiently to release the tightly packed implant 3 (not shown).

FIG. 3C illustrates a soft hinge attachment 20 for the cover flap 7 for the open leading portion of the insertion sheath 1.

FIG. 4 shows an enlargement of the wall of the insertion sheath 1 at the junction of the implant compartment 2 and the tubular handle 4 in the closed position. Illustrated is a “solid pin stabilizer” 21 traversing wall 14 of the implant compartment 2 and the wall 15 of the tubular handle 4 at the junction of these two components of the disclosed invention. The solid pin stabilizer 21 is attached to the pull wire, 10 which when activated, will dislodge the solid pin stabilizer” 21 in each leaf 17 18 19 downward, allowing each leaf 17 18 19 to open, thus releasing the tightly packed implant 3 (not shown in this drawing).

FIG. 4A shows an enlargement of the wall of the insertion sheath 1 at the junction of the implant compartment 2 and the tubular handle 4 in the open position with the pin 21 pulled down by the pull wire 10. The leaf 17 is in a slightly open position and the previously tightly held implant 3 (not shown) is released. The implant 3 is held in position by the disc floor 5 as the insertion sheath 1 is withdrawn.

FIG. 4B illustrates an embodiment of the invention, which uses a circumferential wire 22 to hold each leaf 17 18 19 of the wall of the of the implant compartment 2 in place. When this embodiment of the invention is used, a wire cutting device 23, activated by the downward traction of the pull wire 10, will cut the wire near the distal end of the insertion sheath 1 and allow it to be withdrawn by downward traction of the pull wire 10.

In another an embodiment of the invention, which uses a circumferential wire 22 to hold each leaf 17 18 19 of the wall of the of the implant compartment 2 in place, a cutting apparatus 23A (as described in Details of the Invention and not shown in this drawing), may be attached to a rod that is in a groove on the outside walls 14 15 of the insertion sheath 1. Upward, or downward movement of the rod will cut the circumferential wire 22 and allow it to be withdrawn by downward traction of the pull wire 10 allowing the implant compartment to open.

FIG. 4C illustrates an embodiment of the invention, which uses a circumferential band 24 to hold each leaf 17 18 19 of the wall of the of the implant compartment 2 in place. The band 24 serves as the release mechanism. Downward traction by the pull wire 10 lowers the position of the band 24 and releases each leaf 17 18 19 of the wall 14 of the of the implant compartment 2 thereby releases the tight hold on the implant 3.

What is claimed is:

1. A medical procedure comprising the steps of:
a) identifying an implant site within a patient;
b) creating a cavity within the patient at the implant site;
c) inserting a sheath into the implant site, said sheath having a pre-sterilized and pre-packaged inflatable implant therein, said implant having a fill tube extending therefrom;
d) opening an end of the sheath to expose the implant;
e) withdrawing the sheath from the implant site while maintaining the implant within the surgical site; and,
f) filling the implant via the fill tube.

2. The medical procedure according to claim 1,
a) wherein the fill tube has a self-sealing valve; and,
b) further including the step of, after the step of filling the implant, removing the fill tube from the implant.

3. The medical procedure according to claim 2, further including the step of, after inserting a sheath into the implant site, orienting said sheath within the cavity at the implant site.

4. The medical procedure according to claim 3, further including the step of, before the step of opening an end of the sheath, releasing the implant from said sheath.

5. The medical procedure according to claim 4, wherein the step of opening an end of the sheath includes the step of pulling a wire to dislodge pins holding the implant to the sheath.

6. The medical procedure according to claim 1, wherein the step of withdrawing the sheath includes the simultaneous steps of:
a) pressing a rod onto the implant to maintain the implant within the implant site; and,
b) pulling the sheath from the implant site.

7. The medical procedure according to claim 1, wherein the step of opening an end of the sheath includes the step of opening a hinged distal end of the sheath.

8. A implant procedure comprising the steps of:
a) inserting a sheath into an implant site, said sheath having a pre-sterilized and pre-packaged inflatable implant therein;
b) orienting the sheath in the implant site;
c) opening an end of the sheath to expose the implant; and,
d) withdrawing the sheath from the implant site while maintaining the implant within the surgical site.

9. The implant procedure according to claim 8, further including the step of creating a cavity within the patient at the implant site prior to the step of inserting a sheath into an implant site.

10. The implant procedure according to claim 9,
a) wherein said implant includes a fill tube extending therefrom together with a self-sealing valve; and,
b) further including the step of filling the implant via the fill tube after the step of withdrawing the sheath from the implant site.

11. The implant procedure according to claim 10, further including the step of, after the step of filling the implant, removing the fill tube from the implant.

12. The implant procedure according to claim 11,
a) wherein said implant is secured to the sheath; and,
b) further including the step of releasing the implant from the sheath prior to the step of withdrawing the sheath.

13. The implant procedure according to claim 8, wherein the step of withdrawing the sheath includes the simultaneous steps of:
a) pressing a rod onto the implant to maintain the implant within the implant site; and,
b) pulling the sheath from the implant site.

14. The implant procedure according to claim 8, wherein the step of opening an end of the sheath includes the step of opening a hinged distal end of the sheath.

15. A medical procedure comprising the steps of:
a) identifying an implant site within a patient;
b) creating a cavity within the patient at the implant site;
c) inserting a sheath into the implant site, said sheath having a pre-sterilized and pre-packaged inflatable implant therein, said implant having a fill tube extending therefrom and a self-sealing valve;
d) orienting said sheath within the implant site;
e) opening an end of the sheath to expose the implant;
f) withdrawing the sheath from the implant site while maintaining the implant within the surgical site;
g) filling the implant via the fill tube and the self-sealing valve; and,
h) removing the fill tube from the implant.

16. The medical procedure according to claim 15,
a) wherein said implant is secured to the sheath; and,
b) further including the step of releasing the implant from said sheath.

17. The medical procedure according to claim 16, wherein the step of opening an end of the sheath includes the step of pulling a wire to dislodge pins holding the implant to the sheath.

18. The medical procedure according to claim 15, wherein the step of withdrawing the sheath includes the simultaneous steps of:
a) pressing a rod onto the implant to maintain the implant within the implant site; and,
b) pulling the sheath from the implant site.

19. The medical procedure according to claim 15, wherein the step of opening an end of the sheath includes the step of opening a hinged distal end of the sheath.
